(19) United States
(12) Patent Application Publication
(10) Pub. No.: US 2012/0129760 A1
(43) Pub. Date: May 24, 2012

(54) Modified peptides with antiviral properties and methods for obtaining them

(76) Inventors: Artur Martynov, Kharkov (UA); Boris S. Farber, Brooklyn, NY (US); Sonya Sophya Farber, New York, NY (US)

(21) Appl. No.: 12/931,467

(22) Filed: Feb. 1, 2011

(30) Foreign Application Priority Data
Nov. 22, 2010 (RU) PCT/RU2010/000701

(51) Int. Cl. A61K 38/02; A61P 31/12; C12P 21/06

(52) U.S. Cl. 514/1.8; 435/68.1

(57) ABSTRACT

This invention may be used in human and veterinary medicine for the creation of a drug that is effective perorally in the treatment of many viral infections, such as influenza, herpes, and cytomegalovirus.

Summary of the Invention

Modified peptides with antiviral properties and methods for obtaining them, distinct in that in the capacity of the main active ingredient, a mixture (assembly) of oligopeptides is used that are the products of the hydrolysis of proteins with changes in their molecular charges to the opposite are used, and to obtain them, first a partial hydrolysis of protein-containing raw material is conducted, and then a process of chemical modification of the quantity of oligopeptides obtained with a change in the charge to the molecules is conducted; this is used as an antiviral vehicle for the composition of the oligopeptides obtained. This quantity of modified oligopeptides is capable of slowing down the activity of the heterodimer of b-importin cells and slowing the replication of viruses whose replication cycle depends on the function of the nucleus.

An assembly of modified oligopeptides based on a quasi-life, dynamic, self-organizing system that is effective in the treatment of viral infections such as influenza, herpes, and animal viruses at all stages of the development of the infectious process where other drugs are ineffective. This drug has a wide spectrum of activity and a low level of toxicity; it is accessible for industrial production and effective at any stage of the viral replication cycle that depends on cell nuclei.

### TECHNICAL FIELD

This invention is related to veterinary and human medicine—specifically, to virology—and is intended to treat viral illnesses in humans and animals.

### PREVIOUS LEVEL OF TECHNOLOGY

Viral illnesses make up more than 90% of all registered infectious pathologies. However, there are very few antiviral substances that have been put into production. These substances often have toxic properties and a small spectrum of activity; also, a tolerance effect soon manifests against them in the body. The development of antiviral properties that would not have toxic properties and would be effective in the treatment of a wide spectrum of viral infections is therefore an important task in modern medicine. At present, very few substances are known that would be effective at all stages of a viral infection. Except for interferons and their inductors, substances are not yet known that would simultaneously unite curative and antiviral properties in relation to widespread viral illnesses, such as HIV/AIDS, herpes, influenza, and so on. The best-known drug for flu treatment is Rimantadine. This substance, which only blocks the stage in which the virus penetrates into the cell and the early stage of specific reproduction; it does not work on the pathogenesis of the illness. Long-term use of this drug is impossible, as it has neuro-tropic effects and may cause hallucinations and disrupt brain function due to a slowdown in the passage of impulses along nerve

Leucocytic a-interferon is known to be one of the substances effective in the treatment of influenza. This protein is synthesized in activated human leukocytes. It has the property of evoking resistance to the flu in nasopharyngeal epithelial cells. However, its treatment properties are quite insignificant. It has a low level of effectiveness on the second through sixth day of the flu and is a preventive substance. Recombinant interferons are very expensive and often provoke an allergic reaction. Moreover, with the development of the illness, the effectiveness of interferon therapy declines, while the virus's resistance to the interferon increases.

The nearest prototype of a substance that is being patented is modified proteins and their use for the control of viral infections [1]. These are proteins that have been treated with various anhydrides and acylated substances: albumins, lactoferrin, transferin, and lactalbumin. The authors have also patented the mechanism of action for these proteins: a slowing of viral adhesion. These proteins must have a molecular mass of more than 60000 with a small amount of variance. The significant preventive antiviral activity of these proteins has been demonstrated in experiments on cell cultures. The drugs demonstrated activity against HIV (human and Old World monkey), influenza, cytomegalovirus, the polio virus, the Semliki forest virus, the Sendai virus, the paraflu, and the Coxsackie virus. The authors have proven that acylated proteins are non-toxic and may protect animals from viral infection.

The prototype has some shortcomings: it is strictly a preventive drug (these proteins did not demonstrate a therapeutic effect on cells already infected by viruses) and do not have therapeutic properties for infected animals. In connection with the fact that the prototype is a high-molecular protein, it may only be taken parenterally; the drug is an individual combination and does not demonstrate the production of dynamic, self-organizing systems, and correspondingly, the viruses will quickly adapt to the drug.

### DISCLOSURE OF THE INVENTION

At the basis of the invention is the task of synthesizing modified oligopeptides with anti-viral properties and with a new mechanism of action whose use will allow a significant increase in the effectiveness of the treatment and reduce the treatment times of viral illnesses such as influenza and herpesvirus infections.

The task set is addressed through the synthesis of modified peptides with antiviral proper-ties, distinct in that first a partial hydrolysis of protein-containing raw material is conducted, and then a process of chemical modification of the quantity (assembly) of oligopeptides obtained with a change in the charge to the molecules is conducted. For synthesis, proteins may be used such as: ovalbumin (OA), human seralbumin (HSA), bovine seralbumin (BSA), a mixture of milk proteins (MMP), rabbit seralbumin (RSA), lysozyme (LZ), lactoalbumin (LA), casein (CS), soy protein (SP), a mixture thereof, milk (M), and whole egg white (WEW). For the purposes of enzymatic hydrolysis, pepsin, trypsin, chymotrypsin, papainase, K-proteinase, clostripain, thrombin, thermolysine, and elastase may be used. The synthesized oligopeptides are capable of slowing the activity of the heterodimers of the a-b-importins of the cell, which transport viral polynucleotides from the cytoplasm to the nucleus. Accordingly, the slowing of these transport proteins will lead to the blockage of viruses whose replication depends on cell nucleus functions. In addition, the drug is effective when taken orally.

The modifiers presented in FIG. 1 may be used as acylating agents.

In the experiment, the effectiveness of the drug on influenza and herpes on in vivo and in ovo models was demonstrated, as described below. The authors used an assembly of oligopeptides that were the product of the hydrolysis of proteins or mixtures thereof (egg, milk, etc.), but the molecules' charges were changed to the opposite. “Assembly” is a term from supramolecular chemistry. The objects of supramolecular chemistry are supramolecular assemblies that self-assemble out of their complements—that is, fragments that have geometrical and chemical correspondence—similar to the self-assembly of the most complex three-dimensional structures in a live cell[^{2,3}]

### SHORT DESCRIPTION OF DRAWINGS

FIG. 1. Structures of Chemical Modifiers Applied to Change the Charges of Modified Peptide (MP) Oligopeptide Molecules

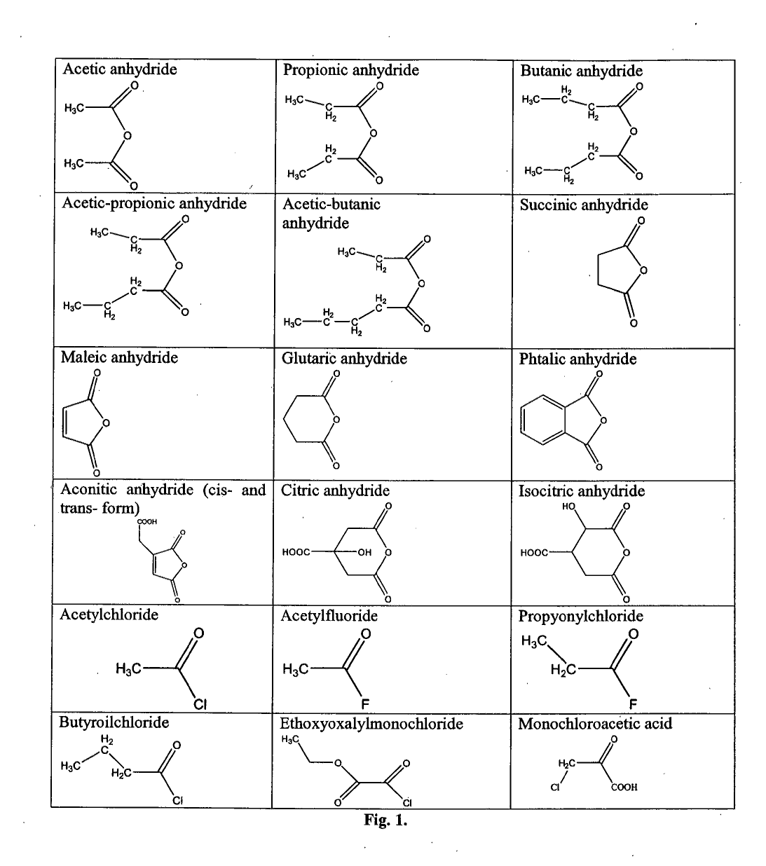

FIG. 2. Micro-Photographs of Infected and Non-Infected Cells Obtained through the Use of a Luminescent Microscope

FIG. 3. Photos of the Eye of a Rabbit Infected with the Type 1 Herpes Virus, and after Treatment (FIG. 3). Wounded Cornea after Introduction of Virus: Hyperemia with “Leukoma” Infection (1, 2) (In the Second Shot, the Wound Location Is Contrasted with Fluorescence). Healthy Cornea after Treatment (3): Hyperemia Is Not Present.

FIG. 4. Electronic Microphotography of Cells Infected with the Herpes Virus: Treated with MP (b) and Not Treated with MP (a)

### BEST INVENTION IMPLEMENTATION OPTION

### Example 1

### Obtaining Mixtures (Compositions) of Modified Peptides (MPs) with Antiviral Properties that are Capable of Self-Organizing into Importins

Under aseptic conditions, 500 mg of ovalbumin is dissolved in 50 ml of distilled water and the pH is brought to 8.0 using 1 M of a sodium hydroxide solution. Trypsin is added, the solution is allowed to sit for 3-45 hours, and the hydrolysis of the ovalbumin with the formation of a peptide mixture is observed. To this mixture, 501-2000 mg of succinic anhydride are mixed for 20 minutes at a temperature of 16-65° C. The mixture is run through membrane filters with the goal of sterilization, and is then poured into glass flagons.

To determine the maximum tolerable concentration (MTC) in the toxological experiments and to study the antiviral activity of the MP drug, the following types of passaged cells of human and animal origin were used:
HS—passaged cells from the kidneys of the embryos of large, horned stock
Tr—passaged cells from the trachea of the embryos of large, horned stock
Hep-2—passaged human larynx cancer cells
Hela—passaged cervical cancer cells
Chicken embryos

The cells were cultured in a 199 medium with the addition of 10% bull blood serum and antibiotics (penicillin and streptomycin). In the capacity of test viruses, the flu virus (H3N2), the vesicular stomatitis virus (Indiana strain), the coronavirus (X 343/44) and the type 1 herpes simplex virus (L-2 strain).

The study was conducted in accordance with the methodology recommended by the State Pharmacological Center of the Ukrainian Ministry of Health.

### Example 2

### A Study of the Toxicity and Determination of the MTC of the MP Drug on Cell Cultures and Chicken Embryos

To determine the MTC, two-day cultures of cells with well-formed cell monolayers were used. The MP drug was tested five separate times on each of the four types of cells listed above. In each experiment, no fewer than 10 test tubes were used for each of the cultures. After removal of the growth medium from the test tubes, 0.2 ml of the experimental solution and 0.8 ml of support culture medium was added to each test tube. The cells were incubated at a temperature of 37° C. over 7-8 days.

Test tubes containing cell cultures to which the drug was not added served as controls.

Calculation of the result was conducted according to the presence or absence of cytopathic activity in the cell when examined under a microscope at ×10. The level of cytotoxic action was determined through changes to the morphology of the cells (cells becoming round or wrinkled, degenerating cells pulling away from the glass) and evaluated according to a four-plus system from + to ++++.

The maximum tolerable concentration was determined by the maximum amount of the substance that could be used without causing cytopathic activity in the cell. For these purposes, various dilutions of the drug at a dosage of 0.2 ml were introduced to the cell cultures.

For a study of toxicity in vivo, the drug was introduced at various doses at a volume of 0.2 ml into the allantoic layer of 9-10-day-old chicken embryos (5 embryos per MP dilution) according to the following method:

10-11-day-old embryos were candled, and a pencil was used to note the location of the air sac on the side opposite to the location of the embryo, where there are fewer blood vessels. The area marked was disinfected with an alcohol and iodine solution; the eggshell was then pierced in that place and 0.1 ml of the material was injected with a tuberculin syringe. In order to reach the allantoic layer, the syringe needle was inserted at a depth of 10-15 mm parallel to the long axis of the egg. After infection, the openings were disinfected again with an alcohol and iodine solution, sealed with paraffin, and placed in an incubator at a temperature of 35-37° C. for 72 hours. Before dissection, the embryos were placed for 18-20 hours in a refrigerator at a temperature of 4° C. for maximum congealing of the blood vessels. After this, the eggs were placed on a tray blunt end up, the shell over the air sac was disinfected with a solution of iodine and 96% ethyl alcohol; then they were punctured and removed with sterile forceps. The cover over the air sac was also removed after having first separated it from the nearby chorion-allantois membrane. After 24 and 48 hours of incubation at a temperature of 37° C., the number of live and normally developing embryos was counted. The calculations of LD₅₀ and MTD were done according to the Kerber method.

As a result of the study on various cultures, it was established that MPs are non-toxic to cell cultures at a dose of more than 50 mg/ml. (To increase the concentration of the drug, it was lyophilized and then diluted to a concentration of 5%. The results of the toxicity study in various cultures are presented in Table 1.

TABLE 1
The Toxicity of MP in Cell Cultures

| No. | Cell Culture | MTC (mg/ml) |
|---|---|---|
| 1 | Pathogen | more than 50 |
| 2 | Tr | —//— |
| 3 | Hep-2 | —//— |
| 4 | Hela | —//— |

The MTC for cell cultures treated with MP comes to more than 50 mg/ml.

### Example 3

### A Study of the Antiviral Activity of the MP Drug on the Influenza A Virus (H3 N2)

Water solutions of MP in various dosages (tenfold dilution) were introduced into 15 chicken embryos in the allantoic layer in a volume of 0.2 ml every 12 hours after introduction of the virus in a working dosage (100 TCD 50/0.2 ml).

Each experiment was accompanied by a control of the test virus in a working dosage. The infected and uninfected (control) embryos were incubated at a temperature of 36° C. over 48 hours. Then the embryos from which the allantoic fluid was removed were dissected. The titration of the virus in the allantoic fluid was conducted via the generally accepted methodology with 1% erythrocytes of human blood type 0(1).

The protection factor (PF) was determined in accordance with [1]. The titer of the virus in the experimental and control groups of chicken embryos is presented in Table 2.

TABLE 2
Effective Concentration of MP in
in ovo Influenza Infection Models

| | | | | Minimum |
|---|---|---|---|---|
| | | Virus Titer | | Effective |
| | Drug | (lg TCD 50/ml) | | Concen- |
| | Concentration | Experi- | | tration (MEC |
| Group | (mg/ml) | ment | Control | mg/ml) |
| Control (injected | — | 12 | 12 | — |
| with a 0.9% saline | | | | |
| solution) | | | | |
| Control Group | 50 ± 5 | 0 | 12 | 0.05 |
| | 5 ± 1 | 0 | 12 | |
| | 0.5 ± 0.05 | 2 | 12 | |
| | 0.05 ± 0.005 | 4 | 12 | |
| | 0.005 ± 0.0005 | 10 | 12 | 5 |

As may be seen in Table 2, the minimum effective concentration of MPs in relation to the influenza virus that fully stops viral synthesis is equal to 0.05 mg/ml. When the dilution of the drug is increased, the effectiveness of the MP declines and has a dose-dependent nature. This fact bears witness to the presence of a direct antiviral effect against the H3N2 virus in the MP drug.

### Example 4

### Study of the Antiviral Activity of the MP Drug on Cytopathic Viruses (Vesicular Stomatitis Virus, the Coronavirus, and HSV-1)

The antiviral activity in relation to this group of viruses was determined in cultures of the abovementioned cells. The reaction was produced in the following manner 0.2 ml each of the corresponding virus in a working dosage (100 TCD₅₀/0.2 ml) was introduced into a two-day rinsed cell culture. 0.8 ml of supporting medium was added. When cytopathic activity was observed in the culture, the MP drug was introduced in various doses. As a control, the same test viruses were used without the drug. The cells were incubated at a temperature of 37° C. Reports on the experiment were done on the third, fifth, and seventh days.

A decline in the virus titer under the influence of the drug being tested of 2 lg or more in comparison with the control was determined to indicate antiviral activity.

The results of the study of the antiviral activity of the MP drug are presented in Table 3.

TABLE 3
Study of the Antiviral Activity of the MP Drug on Vesicular
Stomatitis Virus, the Coronavirus, and HSV-1.

| | | MEC, | Maximum Decline in Titer |
|---|---|---|---|
| Drug | Virus | mg/ml | of the Virus, lg TCD 50/ml |
| MP | VVS | 0.05 | 3.8 |
| | CV | 0.05 | 2.8 |
| | HSV-1 | 0.05 | 4.8 |

As may be seen in Table 3, MPs have antiviral activity and ability to stop the reproduction of all viruses we studied at a concentration of 0.05 mg/ml with a MTC of 50 mcg/ml. The drug's CTI is 1000. Moreover, MP was active in relation to all the viruses studied, while not one comparison drug showed the same kind of activity. Thus the drug is not connected with the specific characteristics of the virus or cell culture, but rather affects mechanisms that all cells have in common.

### Example 5

### A Study of the Antiviral Activity of MP In Vitro in Models of Farm Animal Viruses

The tests were run on 96-lunula plastic panels with viruses of the transmissible gastroenteritis of swine (TGS), strain D-52, with an initial titer of 10^{4.0} TCD50/ml (tissue cytopathic doses) in a test tube culture of piglet testicle cells (PTC) and the diarrhea virus for large horned stock of the Oregon strain with an initial titer of 10⁷⁰ TCD₅₀/ml in a test tube culture of saiga kidney cells (SKC).

In a study of virustatic (inhibiting) activity, the cell cultures were infected with the viruses in doses of 100 and 10 TCDunits/ml and incubated at a temperature of 37° C. MPs were introduced in various doses to the cell cultures (CC) 1-1.5 hours after infection (after the absorption period). Eight titer wells were used for each dilution. After introduction of the sister compounds, the cell cultures were incubated at 37° C. for 72-144 hours until clear evidence of cytopathic activity was found in the virus control.

The cell cultures infected by the virus, inactive CCs, and CCs to which only various concentrations of MPs were introduced served as the control groups. The virustatic activity was determined by the difference in the titers of the viruses in the experimental and control groups.

When virucidal (inactivating) activity was determined in various dosage levels of the solution of sister compounds, they were mixed in various amounts with virus-containing materials and incubated at a temperature of 37° C. over a 24-hour period. The control was the virus-containing material, to which, in addition to the solution of the sister compounds was added a placebo (physical solution) and inactive cell cultures. After contact, the mixtures were titered in parallel with the control. The results were calculated 72-144 hours after incubation at 37° C., after an obvious manifestation of cytopathic activity in the control viruses. The virucidal action was determined by the differences in the titers of the experimental and control group viruses and were expressed in 1 g TCD50.

As a result of the studies conducted, it was established that an MP compound in a concentration of 4000 mcg/ml stopped the reproduction of the TGS virus at 2.75 lg TCD50/ml at an infectious dosage of 100 TCD50/ml and in the same does at 3.75 lg TCDunits/ml at an infectious dosage of 10 TCD50/ml. At a dose of 4000 mcg/mg the TGS virus was inactivated at 2.0 lg TCD50/ml. The MP compound at a dose of 4000 mcg/ml inactivated the diarrhea virus for large horned stock at 3.5 l_{g} TCD50/ml.

When toxicity was studied, it was discovered that MPs at a dose of 4000 mcg/ml were not toxic to either cell culture.

Thus the MP compounds have virustatic (inhibiting) and virucidal (inactivating) activity on the TGS virus and the diarrhea virus in large horned stock; chemical drugs may be created based on these compounds for the treatment and prevention of infectious illnesses of viral etiology.

### Example 6

### A Study of the Antiviral Activity of MP in an Experiment on Animals (Herpes-Virus Kerato-Conjunctivitis/Encephalitis in Rabbits)

The specifics of the experimental system and the level of its adequacy against natural human illness undoubtedly play a decisive role in the evaluation of the effect of antiviral substances on the course of an infection. Experimental herpes infections are of interest in that diseases caused by herpes are widespread and extremely variable in clinical symptomology. The models of experimental herpes on animals are finding increasingly wide application in the study of new antiviral substances.

As is well-known, one of the clinical forms of systemic herpes is herpetic encephalitis, which occurs in guinea pigs, hamsters, rats, mice, rabbits, dogs, and monkeys.

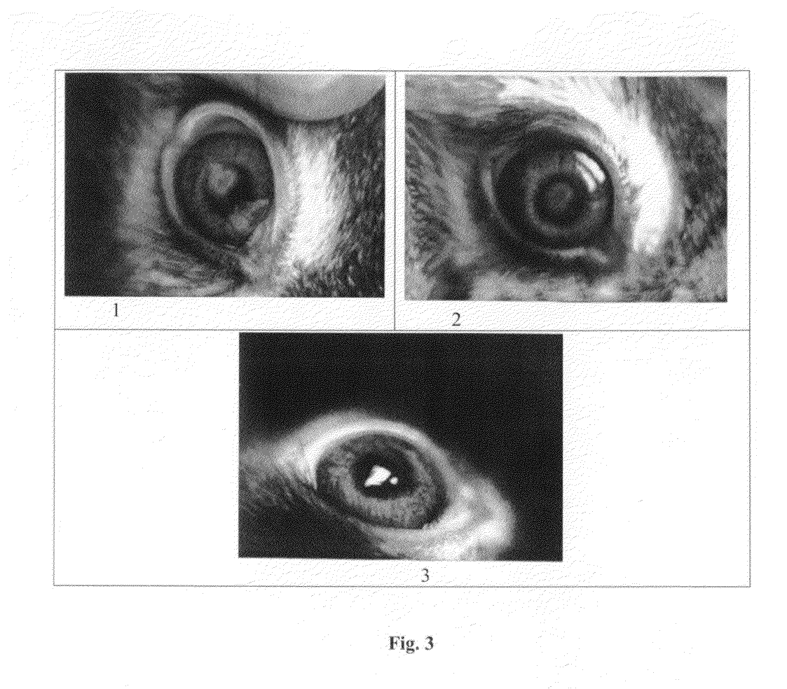

Herpetic keratoconjunctivitis (FIG. 3(1,2)) was caused in rabbits with an average weight of 3.5 kg through introduction of infected material (herpes 1 virus, L-2 strain) into a wounded cornea (FIG. 3(3)). The animal was immobilized and its eye was anesthetized with dicaine (eye drops). The eyelids were pulled back, and several scratches were made on the cornea with a syringe needle. Then the virus-containing material was introduced. The eyelids were closed and rubbed in a circular motion against the cornea. Viral dose: 0.05 ml In the experiment, 16 rabbits were used; of these, 10 were given MPs (daily, beginning on the second day of infection; 14 days at a dosage of 21 mg/kg [which is 7.5 ml of a 1% solution per animal per day]), while six were given a placebo (0.9% sodium chloride).

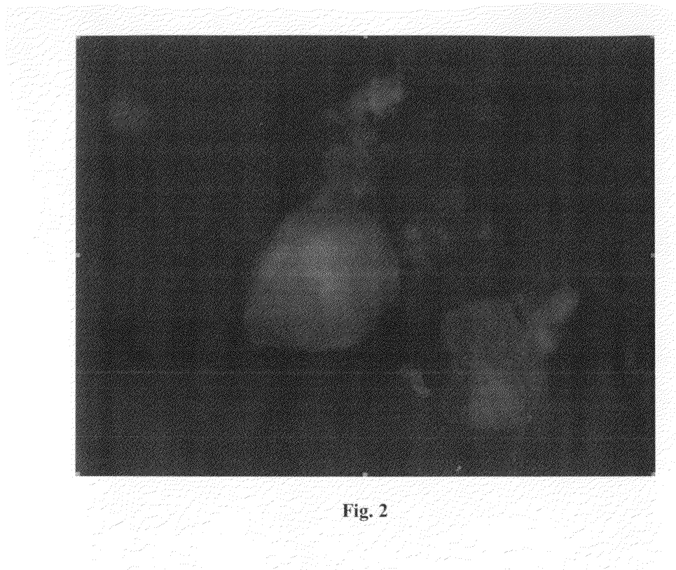

After the rabbits were infected with HSV-1, the condition of their corneas was observed daily for presence of keratoconjunctivitis, encephalitic damage, and presence in the lymphocytes of the peripheral blood of HSV-1 antigens through the immunofluorescence reaction method before and after infection (FIG. 2). Before infection, all the animals' lymphocytes were missing the specific luminescence, which indicated that they did not have antigens to the HSV-1 virus in their peripheral blood. On the third day after infection, the blood of all the animals showed an antigen of HSV-1, IF=70%. In addition, three rabbits (two from the experimental group before treatment and one from the control group) showed encephalitis symptomology: convulsive disorder, loss of appetite. Keratoconjunctivitis developed in all the animals. On the fourth day after infection, the experimental group was administered MPs to the ear vein at a dosage of 21 mg/kg body mass; the control group was administered 0.9% solution of sodium chloride. Over the course of two weeks, this procedure was repeated once a day. In the experimental group, all the animals survived and HSV-1 antigens were not found on the 13th or 14th day. Moreover, in the experimental group, the encephalitis symptoms disappeared by the seventh day of drug administration, whereas in the control group, two animals died. By the 14th day, one animal in the control group had died, while six had died in the control group. Accordingly, the effectiveness indicator was equal to 83.3%, which indicates the high treatment effectiveness of MPs in the model of herpes keratoconjunctivitis/encephalitis in rabbits. In addition, the rabbits in the experimental group gained weight and none showed signs of keratoconjunctivitis. The chemotherapeutic index for rabbits for the MP drug came to 1000, which indicates the promise of MPs as a highly effective antiviral drug with a wide spectrum of activity and low level of toxicity.

### Example 7

### Confirmation of Albuvir's Mechanism of Action

To confirm the MP's mechanism of action, we used DNA from the type 1 L-2 herpes viral strain. They were distinguished as indicated in [⁴]. DNA conjugation with gold particles was conducted according to the method from [⁵]. The compound obtained was introduced into the liposomes according to method [4]. This experiment was described in detail for the SV40 virus in [5].

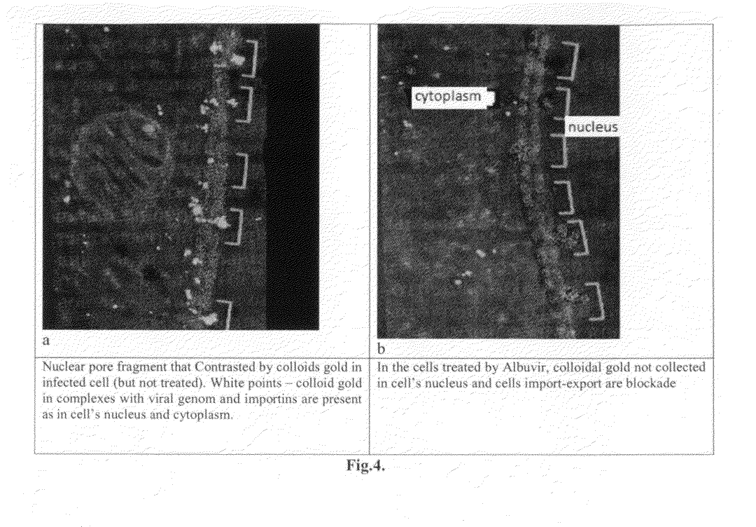

These liposomes merged with the cell membranes from the chicken fibroblast culture. After the merging of the liposome with the cell membrane, the virus's DNA entered the cytoplasm along with the gold particles (FIG. 4a).

The α-β-importin complex carried the colloidal particles into the nuclear pores with the polynucleotide. If the cells were incubated in the presence of the MP, aggregation of the particles of colloidal gold in the nuclear pores was not observed. (FIG. 4b). All the particles were equally distributed throughout the cells' cytoplasm. In this case, the cytopathic activity of the herpes virus was not observed.

Thus the MP slow the process of the transportation of viral DNA to the cell nucleus, which was to be proven.

### Example 8

### The Effectiveness of the MP Drug on Ko66-500 Cross Chickens

The goal of this experiment was the study of the effect of the MP drug on the reproduction of vaccine strains of viruses in the reduction of the titers of the corresponding specific antibodies. It is known that many antiviral drugs, when stopping the reproduction of the live vaccine strains of the viruses lead to the depression of the synthesis of specific antiviral antibodies. This effect is connected with a shortfall in intensity of the infectious process caused by the vaccine in the birds' bodies, and to a weak immune reaction. It is known that in many cases—for example, in infectious bursal disease—the use of live vaccine leads to the induction of the synthesis of such an excessive antibody titer that the bursa becomes exhausted, the bird becomes sensitive to other viruses, and a decrease in weight and increase in mortality occurs. The application of the MP drug should have indicated that it contained antiviral properties according to several parameters: reduction in the excess level of antibody (titers), a decrease in the mortality rate (preservation), and an increase in weight.

For the experiment, 15 chickens per group were used; each was between 36 and 41 days old. The MPs were applied a day before vaccination with live IBD, Gamboro Disease (GB), and infectious bronchitis (IB) vaccines. In the control group were birds that had not been treated with MPs but had been vaccinated. The results of the study are presented in Tables 4 and 5.

TABLE 4
Weight Gain of Chickens (at Time of Slaughter)
in Experimental and Control Groups

| Indicator | Weight Gain**, +% | Survival**, +% |
|---|---|---|
| Experimental Group | 5.2 ± 0.7* | 1.1 ± 0.3* |
| (n = 15) | | |
| Control Group | −1.2 ± 0.3* | −2.1 ± 0.5* |
| (n = 15) | | |

*against the unvaccinated control, which is taken for the base.
**(P = 0.01)

As may be seen in Table 4, in the experimental group, the animals' weight increased by (5.2±0.7) %, while a decrease in weight of (−1.2±0.3) % was observed in the group that was vaccinated but not treated. Also, an increase in survival rates of (1,1±0,3) % was observed in the experimental group.

In Table 5 is presented the change in the titers of specific antiviral antibodies in the group that was treated with MPs and vaccinated, the group that was vaccinated but not treated, and the group that was not vaccinated.

TABLE 5
Changes in the Titer of Antibodies to Infectious Bursal Disease
(IBD), Gamboro Disease (GD), and Infectious Bronchitis (IB)
in Vaccinated Groups and an Un-Vaccinated Control

| | Average Change in the Titer of | | |
|---|---|---|---|
| | Specific Antibodies, ±T | | |
| | IBD | GD | IB |
| Experimental Group | −1000 ± 400 | −600 ± 200 | −1200 ± 400 |
| (vaccinated and | | | |
| treated with MPs) | | | |
| (n = 15) | | | |
| Control Group No. 1 | +2600 ± 700 | +3200 ± 1200 | +2700 ± 1000 |
| (vaccinated, but not | | | |
| treated with MPs) | | | |
| (n = 15) | | | |

| | |
|---|---|
| Control Group | 0 |
| (not treated or | |
| vaccinated) | |

As may be seen in Table 5, the MP has a direct (not immune stimulating) action against all three viruses. The most inhibiting effect was observed in the group with infectious bronchitis: a reduction in the antibody titer by 1200 units. In the vaccinated but untreated control group, the titers of antibodies grew from 2600 units to 3200 units, which indicated that the process of multiplication of the live vaccine in the birds' bodies had been effective.

Thus the application of MPs allows an average of a 5% weight gain in the chickens and a 1% decrease in mortality.

MPs have a direct antiviral action, which stops the reproduction of the viruses that cause infectious bursal disease, Gamboro Disease, and infectious bronchitis.

MPs allow the reasonable restriction of the replication of the vaccine viruses, facilitating a sufficient level of protective antibodies and preventing the exhaustion of the birds' immune systems and the corresponding decrease in weight and increase in mortality.

### Example 9

### The Effect of MPs on the Effectiveness of the Vaccination of Chickens with Live Vaccine

The effect of MPs on the effectiveness of the vaccination was observed directly in an aviaculture business during raising of chickens. When a pathological and anatomical study of the chickens was conducted, characteristic changes were seen for colibacillosis and coccidiosis, as well as many hemorrhages in the mucous membranes of the large intestines and in the transition section between the proventriculus and the gizzard and grinding glands. The contents of the proventriculus were dyed green. The chickens' death rates came to about 15-20% When blood serum of chickens from 38-42 days of age were studied in a hemagglutination inhibition test (HI), specific antibody titers to the Newcastle Disease virus were found that were higher than protective levels (1:1024, 1: 2048).

The study of the effect of MPs at a dosage of 0.03 ml/kg of live weight on the effectiveness of the Newcastle Disease vaccine. For this purpose, one of the aviaries was taken as the control; the others were experimental (Table 6).

TABLE 6
Results of the Study of the Effect of MPs on the
Effectiveness of Vaccination in Aviculture

| | | No. of | |
|---|---|---|---|
| | Aviary | Heads | |
| Group No. | No. | (thousands) | MP Dosage Schedule |
| Control | 4 | 40.0 | MP Not Given |
| Experiment 1 | 8 | 40.0 | From an age of seven days over |
| | | | the course of the three days |
| | | | before vaccination with live |
| | | | viral vaccine |
| Experiment 2 | 7 | 40.0 | 1 Day before Newcastle |
| | | | Disease Vaccination |
| Experiment 3 | 5 | 40.0 | Over the 3 Days before |
| | | | Vaccination and 7-10 Days after |
| | | | Newcastle Disease Vaccination |

The conditions of observation, the microclimate parameters, the lighting regime, the amount of floor space per bird, and the feeding schedule were identical throughout all groups in accordance with the methodological recommendations for raising ROS 308 crosses.

The immune system load was determined at an age of 42 days through HI. Simultaneously, the clinical condition of the birds, their retention rate, their growth, and food loss were calculated.

The results of the experiments for the determination of the effectiveness of MPs when vaccinating chickens against Newcastle Disease are presented in Table 7.

TABLE 7
The Effect of MPs on the Effectiveness
of the Newcastle Disease Vaccine

| | | Experiment | Experiment | Experiment |
|---|---|---|---|---|
| Indicators | Control | 1 | 2 | 3 |
| Average | 21 ± 7.55 | 39.0 ± 15.30 | 84.5 ± 29.39 | 124.0 ± 31.09 ** |
| Titer, | | | | |
| in HI | | | | |
| Immune | 75 | 87.5 | 100 | 100 |
| System | | | | |
| Stress, % | | | | |

Notes:
Reliability in comparison with the control:
* P < 0.05,
** P < 0.01
*** P < 0.001

The average titer of specific antibodies to the Newcastle Disease virus were on the protective level in both the control and experimental groups. However, when the 42-day-old chickens' serum was studied, the ones with MPs used established a significant increase in the average titer in experimental group 3 in comparison with the control group by a factor of 6 (<0.01). In the experimental groups (1, 2), a reliable difference in the antibody titers in comparison to the control could not be established; however, they were on the protective levels, and a tendency to increase this indicator by factors of 1.8 and 4.3 was discovered. The group immunity in the control came to 75%, while it was 100% in experimental groups 3 and 4 and 87.5% in another experimental group. The death rate of the chickens in the control group was 9.8%, while the death rates fell in the experimental groups by a factor of 2.8, 3.3, and 4 respectively in comparison with the control. The average daily growth of the chickens in the experimental groups fluctuated from 52-54 g, while the growth in the control group was 48 g.

Thus a conclusion may be drawn that the optimum scheme for the use of MPs for chickens in regions with complex epizootic situations with Newcastle Disease is the use of the drug at a dosage of 0.03 ml/kg of live weight over the course of 3 days before vaccination and 7-10 days after vaccination against Newcastle Disease. The use of the drug according to the abovementioned scheme will lead to an increase in the average titer of specific antibodies to the Newcastle Disease virus by a factor of 6 and a decrease in the death of the chickens by a factor of 4.

### INDUSTRIAL APPLICABILITY

This invention is related to veterinary and human medicine—specifically, to virology—and may be used for the creation of new drugs on the basis of dynamic, self-adapting and self-organizing systems for the treatment of viral infections in animals and humans. The drugs obtained in this manner are completely ecologically safe, biodegradable, and fully metabolized both in patients' bodies and in the environment; the technology required to make them is completely without waste. The production of the product being patented can be done on the existing equipment of pharmaceutical companies and does not require the development of new, unique equipment; it does not require an expenditure of energy and is waste-free and ecologically clean.

### REFERENCES

1 Robert Walter Jansen, Dirk Klaas Fokke Meijer, Grietje Molema, Erik Desire Alice De Clercq, Rudi Wilfried Jan Pauwels, Dominique Schols Modified proteins and their use for controlling viral infection//A61K 3804; A61K 3816, U.S. Pat. No. 5,869,457, Reg. Sep. 2, 1999. Appl. Sep. 4, 1997
2 http://dic.academic.ru/dic.nsf/ruwiki/79240
3 Jean-Marie Lehn. Supramolecular Chemistry. Concepts and Perspectives.—Weinheim; New York; Basel; Cambridge; Tokyo: VCH Verlagsgesellschaft mbH, 1995.—P. 103 (Chapter 7)
⁴ Feldherr C., Kaltenbach E, Schultz N.//J. Cell. Biol.—1984.—Vol. 99, P. 2216-2222
⁵ Lanford R. E., Butel J. S. Cell.—1984. Vol. 37., P. 801-813

1. Modified peptides with antiviral properties distinct in that in the capacity of peptides, a mixture (assembly) of oligopeptides are used that are products of the hydrolysis of proteins with molecules changed to the opposite charge.

2. Modified oligopeptides with antiviral properties according to claim 1, distinct in that the charge of the molecules are changed through the formation of an amino group as a result of an acylation reaction between di- and tri-carboxylic acids and the remains of lysine and histidines in the oligopeptide mixture with the creation of new carboxyl groups.

3. Modified oligopeptides with antiviral properties according to claim 1, distinct in that the charge of the molecules are changed through the formation of a mixed amino group as a result of an alkylation reaction between monochloracetic acid and the remains of lysine and histidines in the oligopeptide mixture with the creation of new carboxyl groups.

4. A method of obtaining modified peptides with antiviral properties distinct in that to obtain them, first a partial hydrolysis of protein-containing raw material is conducted, and then a process of chemical modification of the quantity (assembly) of oligopeptides obtained with a change in the charge to the molecules is conducted; this is used as an antiviral vehicle for the composition of the oligopeptides obtained.

5. A method of obtaining modified peptides with antiviral properties according to claim 4, distinct in that as a protein-containing raw material, an individual protein is used.

6. A method of obtaining modified peptides with antiviral properties according to claim 4, distinct in that as a protein-containing raw material, a mixture of proteins is used.

7. A method of obtaining modified peptides with antiviral properties according to claim 4, distinct in that as a protein-containing raw material, milk is used.

8. A method of obtaining modified peptides with antiviral properties according to claim 4, distinct in that as a protein-containing raw material, initial egg albumen is used.

9. A method of obtaining modified peptides with antiviral properties according to claim 4, distinct in that for the partial hydrolysis of protein-containing raw material, enzymatic hydrolysis is used.

10. A method of obtaining modified peptides with antiviral properties according to claim 4, distinct in that for the partial hydrolysis of protein-containing raw material, acidic hydrolysis is used.

11. A method of obtaining modified peptides with antiviral properties according to claim 4, distinct in that for the partial hydrolysis of protein-containing raw material, alkaline hydrolysis is used.

12. A method of obtaining modified peptides with antiviral properties according to claim 4, distinct in that for the partial hydrolysis of protein-containing raw material, synthetic peptidases are used.

13. A method of obtaining modified peptides with antiviral properties according to claim 4, distinct in that for the chemical modification of an amount of oligopeptides, succinic anhydride is used.

14. A method of obtaining modified peptides with antiviral properties according to claim 4, distinct in that for the chemical modification of an amount of oligopeptides, monochloracetic acid is used.
